(19) United States
(12) Patent Application Publication
(10) Pub. No.: US 2024/0018468 A1
(43) Pub. Date: Jan. 18, 2024

(54) SURFACE ACOUSTIC WAVE (SAW) 3D PRINTING METHOD

(71) Applicant: AO TECHNOLOGY AG, Chur (CH)

(72) Inventors: Tiziano SERRA, Davos Platz (CH); David Olivier EGLIN, Davos Frauenkirch (CH); Mauro ALINI, Davos Platz (CH)

(73) Assignee: AO TECHNOLOGY AG, Chur (CH)

(21) Appl. No.: 18/363,407

(22) Filed: Aug. 1, 2023

Related U.S. Application Data
(63) Continuation of application No. 16/641,526, filed on Feb. 24, 2020, now abandoned.

(30) Foreign Application Priority Data
Aug. 25, 2017 (CH) 01058/17

(51) Int. Cl. C12N 5/00; B33Y 10/00; B29C 64/188; B29C 64/165; B33Y 70/10; C12M 1/12; C12N 11/04

(52) U.S. Cl. CPC C12N 5/0062; B33Y 10/00; B29C 64/188; B29C 64/165; B33Y 70/10; C12M 25/14; C12N 11/04; B29K 2089/00; B29K 2105/0061

(57) ABSTRACT

A process for the production of a three-dimensional particulate structure embedded in a body formed of a hydrogel matrix, comprising the steps of forming a layer of a hydrogel matrix having a particulate substructure embedded therein by subjecting a suspension of particulates in a layer of a hydrogel matrix precursor to standing acoustic waves (SAWs) to spatially partition the particulates within the layer of hydrogel precursor into a particulate substructure and allowing the hydrogel precursor to solidify such as to form the layer of a hydrogel matrix having a particulate substructure embedded therein, and repeating the process until the three-dimensional particulate structure embedded in a body formed of a hydrogel matrix is formed.

### CROSS REFERENCE TO RELATED APPLICATIONS

This application is a Continuation of U.S. application Ser. No. 16/641,526, filed on Feb. 24, 2020, which is a National Stage of International Application No. PCT/EP2018/073028 filed Aug. 27, 2018, claiming priority based on Swiss Patent Application No. 01058/17 filed Aug. 25, 2017, the contents of all of which are incorporated herein by reference in their respective entireties.

### TECHNICAL FIELD

The present invention relates to an additive manufacturing process for obtaining three-dimensional particulate structures embedded in a body formed of a hydrogel matrix.

### BACKGROUND

State-of-the-art manufacturing technologies for three-dimensional constructs including live cells either requires the development of very specific bio-inks or are based on manipulation/deposition of single cells on scaffolds, which is a lengthy process when large constructs or numerous constructs need to be produced.

Acoustic waves have been known to be useful for the positioning of cells in liquid media than can be crosslinked, which allows obtaining roughly two-dimensional constructs including live cells and/or bioactive particles very rapidly. The positioning of cells in a liquid medium exposed to acoustical waves is nearly instantaneous, so the time needed to fix the cells and/or bioactive particles within a cross-linkable medium is mainly determined by the time the cross-linkable medium needs to solidify. However, when using standing acoustic waves, it is only possible to orient cells in a roughly two dimensional manner, since the partitioning of the cells will be governed by the position of the nodes and anti-nodes on the surface of the liquid layer. As an example, it is not possible to form a structure that varies along the z-direction, i.e. a direction perpendicular to the surface of the liquid medium such as for example a sphere or a cone. While such structures may for example be formed by 3D printing techniques, these techniques suffer from the drawback that they are relatively time consuming and require special bioinks and 3D printing apparatuses. In addition, the shear forces experienced by the cells during extrusion of the bioinks through the nozzle of the printing apparatus decrease the viability of the cells.

WO 2016/069493 A2 relates to a method of making a multi-layer patent cell assembly in which a cells suspension liquid solution containing cells is loaded into a liquid-carrier chamber. Once the cells in the cells suspension liquid solution have gravitationally settled down to the bottom of the chamber, a hydrodynamic drag force in the form of so-called Faraday waves is applied to a vibration generator such that the settled cells are oriented into a certain distribution.

WO 2015/112343 A1 provides for a system and method for providing tissue regeneration without the use of a scaffold. The system includes a vessel that contains a fluid suitable for enhancing the tissue regeneration process, as well as an acoustic transducer at one end of the vessels and a reflector at an opposite end of the vessel. The transducer provides an acoustic signal that creates standing acoustic fields in the vessel that confine cells within the fluid into a plurality of structures.

WO 2013/118053 A1 relates to a method of forming a multi-layer aggregates of objects such as cells in a channel comprising a liquid where the aggregates are formed by applying acoustics waves such as stationary waves within each region onto said objects.

US 2004/0137163 A1 provides for a system and a method for robotic manipulation of objects where in a liquid agitated by the transfer of energy thereto, such as for example by vibration, standing waves are formed which align the objects along nodes of the standing waves. The location of the standing waves can be determined by controlling the energy input, by variation of the size and shape of the container.

There is thus a need for an additive manufacturing process in which large-volume constructs can be achieved in a time-effective manner and in sufficient complexity, and in which cell viability is retained.

### SUMMARY OF THE INVENTION

The above problem has been solved in the present invention by providing a process which allows preparing complex three-dimensional structures with a less complicated apparatus and at the same time reducing the time required to provide such complex three-dimensional structures.

It is an object of the present invention to provide a process for the production of a three-dimensional particulate structure embedded in a body formed of a hydrogel matrix, comprising the steps of a. forming a layer of a hydrogel matrix having a particulate substructure embedded therein by i. providing a suspension of particulates in a layer of a hydrogel matrix precursor in a container, said container having one or more inner surface portions vibrationally coupled to one or more vibration generators; ii. subjecting the suspension of particulates in the layer of a hydrogel matrix precursor to a vibration emanating from at least the inner surface portion of the container vibrationally coupled to the vibration generator such as to cause standing acoustic waves in the hydrogel matrix precursor, thereby spatially partitioning the particulates within the layer of hydrogel precursor into a particulate substructure or structure; iii. allowing the hydrogel precursor to solidify such as to form the layer of a hydrogel matrix having a particulate substructure or structure embedded therein. Thus, the present invention provides a process in which two or more different types of particulates can be differently partitioned within a single layer of a hydrogel matrix using standing acoustic waves.

In a preferred embodiment, the process according to the present invention further includes the step of b. forming a further layer of a hydrogel matrix having a particulate substructure embedded therein by carrying out step a. in a separate container and depositing said further layer on top of the previously formed layer of a hydrogel matrix having a particulate substructure embedded therein; in particular such as to form a three-dimensional particulate structure embedded in a body formed of a hydrogel matrix, and/or eventually c. repeating step b. at least one, two, three or more times such as to form a three-dimensional particulate structure embedded in a body formed of a hydrogel matrix.

It is another object of the present invention to provide a process for the production of a three-dimensional particulate structure embedded in a body formed of a hydrogel matrix, comprising the steps of a. forming a layer of a hydrogel matrix having a particulate substructure embedded therein by i. providing a suspension of particulates in a layer of a hydrogel matrix precursor in a container, said container having one or more inner surface portions vibrationally coupled to one or more vibration generators; ii. subjecting the suspension of particulates in the layer of a hydrogel matrix precursor to a vibration emanating from at least the inner surface portion of the container vibrationally coupled to the vibration generator such as to cause standing acoustic waves in the hydrogel matrix precursor, thereby spatially partitioning the particulates within the layer of hydrogel precursor into a particulate substructure; iii. allowing the hydrogel precursor to solidify such as to form the layer of a hydrogel matrix having a particulate substructure embedded therein; b. forming a further layer of a hydrogel matrix having a particulate substructure embedded therein by carrying out step a. in a separate container and depositing said further layer on top of the previously formed layer of a hydrogel matrix having a particulate substructure embedded therein; c. repeating step b. at least one, two, three or more times such as to form a three-dimensional particulate structure embedded in a body formed of a hydrogel matrix.

### SHORT DESCRIPTION OF THE DRAWINGS

Preferred embodiments of the invention are described in the following with reference to the drawings, which are for the purpose of illustrating the present preferred embodiments of the invention and not for the purpose of limiting the same. In the drawings,

FIG. 1 shows pictures of particulate structures embedded in a body formed of a hydrogel matrix (a,b,c), which confirm the simulation prediction of particulate segregation. The corresponding simulation predictions are shown in perspective view (d,e,f) as well as from the top (g,h,i).

FIG. 2 shows an apparatus for creating standing acoustical waves and different parts thereof (a, b, c). In FIGS. 2d-2i fluorescent microscopy images of patterned hMSCs obtained using a frequency of 158 Hz and Amplitude=6 V approx. are shown. The obtained pattern forms concentric circles, as can be seen from FIG. 1d. FIGS. 2e-2i show higher magnification fluorescent microscopy images, in which nuclei are stained with DAPI and actin cytoskeleton are stained with phalloidin.

FIG. 3 shows a GelMA/TCP microparticle suspension having a rounded checkerboard-like shape of TCP microparticles embedded in GelMA (a, b), shows a GelMA/iron oxide nanoparticles suspension displaying an continuous and homogenous layer of iron oxide nanoparticles embedded in GelMA (c), shows a GelMA/TCP microparticle suspension having a concentric circle shape of TCP microparticles embedded in GelMA (f, h), as well as the superposition of the respective layers (d,i)

FIG. 4 shows a schematic descriptive of the superposition of three layers of GelMA hydrogel

FIG. 5 shows images of three samples obtained in which TCP particles having a diameter in the range of 32 to 75 μm (white particles) are differently partitioned than resin particles having a diameter in the range of 37 to 74 μm (grey). Circular empty spaces are appear black.

FIG. 6 shows a fluorescence image of a sample obtained in which TCP particles having a diameter in the range of 250 to 500 μm (black particles) form quasi-circles, and in which quasi-circles hMSC spheroids agglomerate (grey particles).

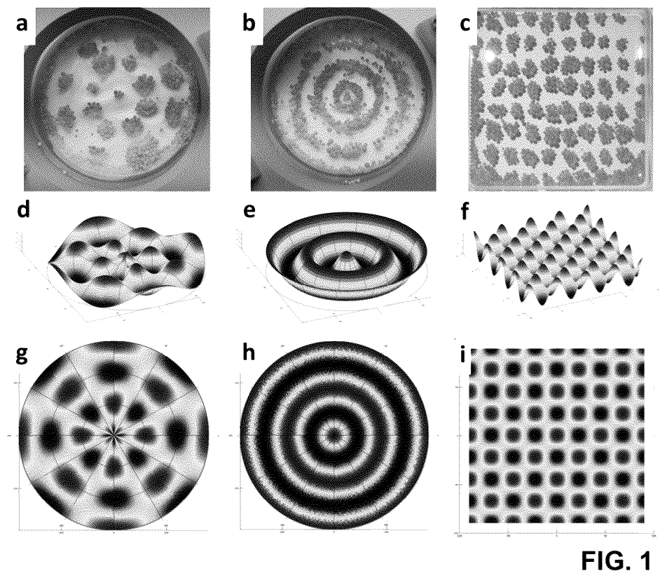

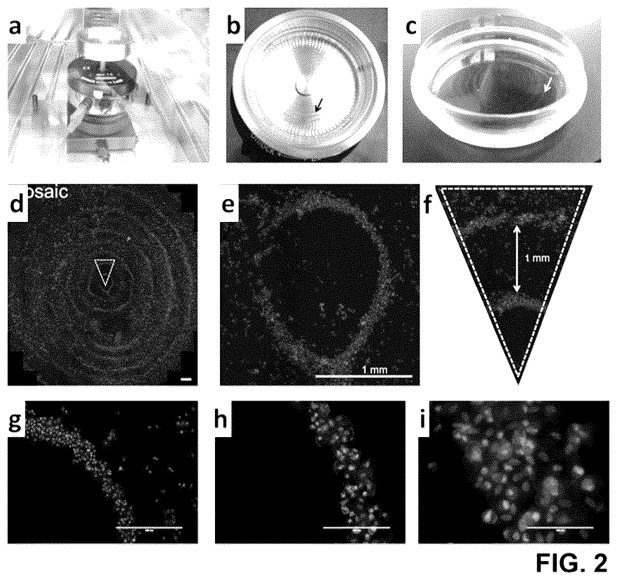

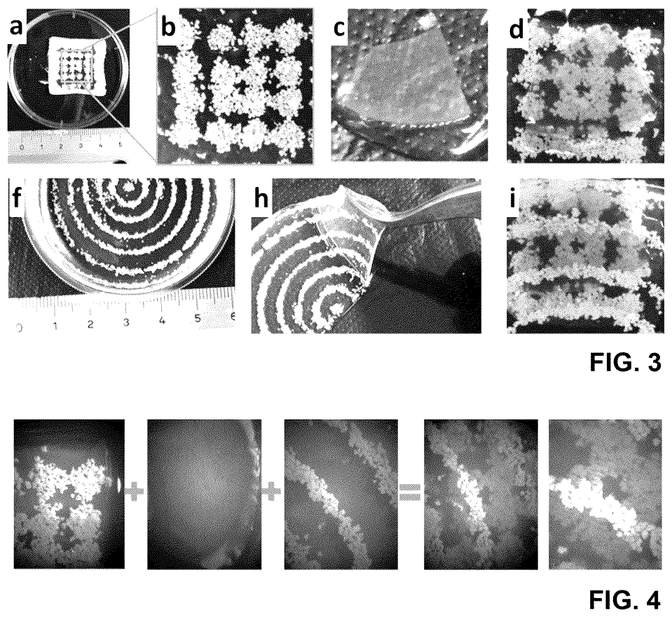

### PREFERRED EMBODIMENTS

It is an object of the present invention to provide a process for the production of a three-dimensional particulate structure embedded in a body formed of a hydrogel matrix, comprising the steps of a. forming a layer of a hydrogel matrix having a particulate substructure embedded therein by i. providing a suspension of particulates, preferably a suspension of two or more different types of particulates, in a layer of a hydrogel matrix precursor in a container, said container having one or more inner surface portions vibrationally coupled to one or more vibration generators; ii. subjecting the suspension of particulates, preferably a suspension of two or more different types of particulates, in the layer of a hydrogel matrix precursor to a vibration emanating from at least the inner surface portion of the container vibrationally coupled to the vibration generator such as to cause standing acoustic waves in the hydrogel matrix precursor, thereby spatially partitioning the particulates, preferably differently spatially partitioning the two or more different types of particulates, within the layer of hydrogel precursor into a particulate substructure or structure; iii. allowing the hydrogel precursor to solidify such as to form the layer of a hydrogel matrix having a particulate substructure or structure embedded therein.

In a preferred embodiment, the process according to the present invention further includes the step of b. forming a further layer of a hydrogel matrix having a particulate substructure embedded therein by carrying out step a. in a separate container and depositing said further layer on top of the previously formed layer of a hydrogel matrix having a particulate substructure embedded therein; in particular such as to form a three-dimensional particulate structure embedded in a body formed of a hydrogel matrix, and/or eventually c. repeating step b. at least one, two, three or more times such as to form a three-dimensional particulate structure embedded in a body formed of a hydrogel matrix.

It is another object of the present invention to provide a layer-by-layer process for the production of a three-dimensional particulate structure embedded in a body formed of a hydrogel matrix, comprising the steps of a. forming a layer of a hydrogel matrix having a particulate substructure embedded therein by i. providing a suspension of particulates in a layer of a hydrogel matrix precursor in a container, said container having one or more inner surface portions vibrationally coupled to one or more vibration generators; ii. subjecting the suspension of particulates in the layer of a hydrogel matrix precursor to a vibration emanating from at least the inner surface portion of the container vibrationally coupled to the vibration generator such as to cause standing acoustic waves in the hydrogel matrix precursor, thereby spatially partitioning the particulates within the layer of hydrogel precursor into a particulate substructure; iii. allowing the hydrogel precursor to solidify such as to form the layer of a hydrogel matrix having a particulate substructure embedded therein; b. forming a further layer of a hydrogel matrix having a particulate substructure embedded therein by carrying out step a. in a separate container and depositing said further layer on top of the previously formed layer of a hydrogel matrix having a particulate substructure embedded therein; c. repeating step b. at least one, two, three or more times such as to form a three-dimensional particulate structure embedded in a body formed of a hydrogel matrix.

In the process for the production of a three-dimensional particulate structure embedded in a body formed of a hydrogel matrix according to the present invention, the three-dimensional particulate structure may have any form since the process has no limitations with respect to the three-dimensional particulate structure except for the resolution of the structure in z-direction, i.e. a direction perpendicular to the surface of the hydrogel precursor, which is of course dependent on the thickness of the individual layers of hydrogel matrix precursor. While the thickness of the layer is typically in the micrometre range, e.g. 50 to 500 micrometres, the process for the production of a three-dimensional particulate structure embedded in a body formed of a hydrogel matrix according to the present invention may be up to 10 or even 15 mm, thereby allowing the fast production of a complex three-dimensional particulate structure embedded in a body formed of a large volume of hydrogel matrix. Exemplary three-dimensional particulate structures embedded in a body formed of a hydrogel matrix can be spheres, closed cylinders, cones and such. The containers suitable in the process for the production of a three-dimensional particulate structure embedded in a body formed of a hydrogel matrix according to the present invention may be of any shape and material, provided that they are capable of effectively transmitting the vibrations from the vibration generator to the hydrogel matrix precursor in the container. Exemplary containers are for example Petri dishes of polymer or glass.

In the process for the production of a three-dimensional particulate structure embedded in a body formed of a hydrogel matrix according to the present invention, the particulate structure is embedded in a body formed of a hydrogel matrix. It is understood that the particulate structure may be formed of one type of particulates in its entirety or may be formed of different types of particulates. It is further understood that it will be within the reach of the person skilled in the art to choose the concentration, as well as the type of particulates, in each layer of a hydrogel matrix to arrive at a desired overall three-dimensional particulate structure embedded in a body formed of a hydrogel matrix. Furthermore, it is understood that these considerations will apply likewise to the type of hydrogel matrix in each layer, which may be changed at each layer-forming iteration of the process.

In the process for the production of a three-dimensional particulate structure embedded in a body formed of a hydrogel matrix according to the present invention, the three-dimensional particulate structure is obtained by forming multiple layers of a hydrogel matrix having a particulate substructure embedded therein and by superposing them such as to form a three-dimensional particulate structure embedded in a body formed of a hydrogel matrix.

The layers forming the three-dimensional particulate structure embedded in a body formed of a hydrogel matrix are formed by providing a suspension of particulates in a layer of a hydrogel matrix precursor in a container, said container having one or more inner surface portions vibrationally coupled to one or more vibration generators; subjecting the suspension of particulates in the layer of a hydrogel matrix precursor to a vibration emanating from at least the inner surface portion of the container vibrationally coupled to the vibration generator such as to cause standing acoustic waves in the hydrogel matrix precursor, thereby spatially partitioning the particulates within the layer of hydrogel precursor into a particulate substructure or structure; and allowing the hydrogel precursor to solidify such as to form the layer of a hydrogel matrix having a particulate substructure or structure embedded therein.

The suspension of particulates in a layer of a hydrogel matrix precursor in a container can be provided by previously preparing a suspension of particulates in a hydrogel matrix precursor and dosing a predetermined volume of it into the container. The suspension of particulates in a hydrogel matrix precursor may be prepared by agitating a mixture of particulates and hydrogel matrix precursor such as to preferably obtain a isotropic spatial distribution of the particulates within the hydrogel matrix precursor. In the case where the particulates are cells, it is preferable to agitate the mixture in a manner that additionally does not result in a decrease in viability of the cells such as to take full advantage of the conservation of viability deriving from the use of standing acoustic waves to partition the cells. In order to generate the standing acoustic waves, the container holding the suspension of particulates in a hydrogel matrix precursor has one or more inner surface portions vibrationally coupled to one or more vibration generators. This allows to transmit vibrations leading to the generation of standing acoustic waves to the suspension of particulates in a hydrogel matrix precursor and to achieve the partitioning of the particulates within the hydrogel matrix precursor. Once the standing acoustic wave is formed, the particulates will spatially segregate such as to concentrate under the nodal regions of the standing acoustic waves and thus leaving the anti-nodal regions free of particulates. Once the particulates have spatially segregated, the hydrogel matrix precursor is allowed to solidify such as to form the layer of a hydrogel matrix having a particulate substructure or structure embedded therein. By solidifying the layer of hydrogel matrix precursor, the particulates are fixed spatially and embedded in the continuous phase of hydrogel matrix, and the process of forming the next layer of a hydrogel matrix having a particulate substructure embedded therein can be repeated multiple times until the final three-dimensional particulate structure embedded in a body formed of a hydrogel matrix is obtained. Thus, the final three-dimensional particulate structure embedded in a body formed of a hydrogel matrix is basically formed by stacking pre-fabricated layers of particulate substructures embedded in a layer of hydrogel matrix.

In a preferred embodiment of the process for the production of a three-dimensional particulate structure embedded in a body formed of a hydrogel matrix according to the present invention, the hydrogel precursor is allowed to solidify by partially crosslinking the hydrogel precursor. It is understood that “partially crosslinking the hydrogel precursor” means that essentially the hydrogel precursor in the respective layer is partially and evenly crosslinked throughout the bulk of the layer such as to yield one uninterrupted layer of solidified hydrogel precursor embedding the particulates. By merely partially crosslinking the hydrogel matrix precursor, the solidified layer of a hydrogel matrix having a particulate substructure embedded therein retains a part of its crosslinking ability. Therefore, when subsequent layers of a hydrogel matrix having a particulate substructure embedded therein are deposited on top of the previous, partially cross-linked layer(s), the previous layer(s) and the last layer can cross-link between them. In this manner, a three-dimensional particulate structure embedded in a body formed of a hydrogel matrix is formed that has increased mechanical properties because the layers making up the body are bound to each other and thus cannot slide laterally with respect to each other. In a preferred embodiment of the process for the production of a three-dimensional particulate structure embedded in a body formed of a hydrogel matrix according to the present invention, the hydrogel precursor is thus allowed to is solidify by partially crosslinking the hydrogel precursor by exposing it to 60%, 70% or 80% or from 60% to 80% of the radiation dose needed to fully crosslink the hydrogel precursor, in the case where the crosslinking agent is capable of being activated by radiation. In a more preferred embodiment of the process for the production of a three-dimensional particulate structure embedded in a body formed of a hydrogel matrix according to the present invention, the partially crosslinked layers of a hydrogel matrix having a particulate substructure embedded therein forming the three-dimensional particulate structure embedded in a body formed of a hydrogel matrix are fully crosslinked in an additional step to yield a three-dimensional particulate structure embedded in a body formed of a hydrogel matrix that has better mechanical properties and in which the individual layers of a hydrogel matrix having a particulate substructure embedded therein adhere to each other. In the context of the present invention, “solidify” means that a substance is self-supporting.

In a preferred embodiment of the process for the production of a three-dimensional particulate structure embedded in a body formed of a hydrogel matrix according to the present invention, the hydrogel precursor is allowed to solidify by partially crosslinking the hydrogel precursor, and said partial cross-linking is achieved by using a crosslinking agent that does not cross-link instantaneously upon being activated. When a further layer of a hydrogel matrix having a particulate substructure embedded therein is deposited on the previous layer before the cross-linking of the previous layer of a hydrogel matrix having a particulate substructure embedded therein is complete, an increased bonding of the layers is achieved which results in a three-dimensional particulate structure embedded in a body formed of a hydrogel matrix which is mechanically resistant.

In a preferred embodiment of the process for the production of a three-dimensional particulate structure embedded in a body formed of a hydrogel matrix according to the present invention, the hydrogel precursor is solidified by partially crosslinking the hydrogel precursor using a crosslinking agent, preferably by a crosslinking agent capable of being activated by a physical stimulus such as radiation or temperature change or by a chemical stimulus such as enzymes, pH change or ion concentration change. In the case crosslinking agent capable of being activated by a physical stimulus such as radiation or temperature change is used, a heating/cooling system controlling the temperature of the hydrogel layer or a Hg vapour or LED lamp capable of irradiating the hydrogel layer can be used. In the case crosslinking agent capable of being activated by a chemical stimulus is used, the chemical stimulus can be delivered by a spray gun to the hydrogel layer.

In a preferred embodiment of the process for the production of a three-dimensional particulate structure embedded in a body formed of a hydrogel matrix according to the present invention, the hydrogel matrix comprises gelatine methacrylate or hyaluronic acid methacrylate. Alternatively, the hydrogel matrix may further comprise gelatine, collagen, fibrin/thrombin, MATRIGEL®, agarose, hyaluronan tyramine, gelatine tyramine, alginate or other hydrogels known in the art that are preferably suitable for use in biomedical applications.

In a preferred embodiment of the process for the production of a three-dimensional particulate structure embedded in a body formed of a hydrogel matrix according to the present invention, the particulates are inorganic particulates, in particular inorganic particulates capable of supporting bio-mineralization in an implant, such as hydroxyapatite particulates or calcium phosphate.

In a preferred embodiment of the process for the production of a three-dimensional particulate structure embedded in a body formed of a hydrogel matrix according to the present invention, the particulates are organic particulates, in particular organic particulates capable of forming a scaffold in a medical implant, such as polylactic acid or polyhydroxybutyric acid

In a preferred embodiment of the process for the production of a three-dimensional particulate structure embedded in a body formed of a hydrogel matrix according to the present invention, the particulates are cells or aggregates of cells or cell spheroids. In particular, the cells may be animal cells such as osteoblasts, fibroblasts, keratinocytes, human mesenchymal stem cells (hMSCs), chondrocytes or human umbilical vein endothelial cells (hUVECs).

In a preferred embodiment of the process for the production of a three-dimensional particulate structure embedded in a body formed of a hydrogel matrix according to the present invention, the particulates are organic particulates of two or more different types. It is understood that in the context of the present invention, different types of particles are in general types of particles that spatially partition differently when exposed to the same standing acoustic waves. Exemplary differences in particle types may be different with respect to densities, to geometries, to chemical composition, to particle size, cell type, and combinations thereof.

In a preferred embodiment of the process for the production of a three-dimensional particulate structure embedded in a body formed of a hydrogel matrix according to the present invention, the inorganic particulates are capable of supporting bio-mineralization in an implant, such as for example hydroxyapatite or calcium phosphate particulates and/or wherein the organic particulates are capable of forming a scaffold in a medical implant, such as for example polylactic acid or polyhydroxybutyric acid.

In a preferred embodiment of the process for the production of a three-dimensional particulate structure embedded in a body formed of a hydrogel matrix according to the present invention, the particulate substructure or structure of a layer is formed such as to have an identical, similar or preferably differing particulate distribution as, or preferably from, a particulate substructure or structure of another layer.

In a preferred embodiment of the process for the production of a three-dimensional particulate structure embedded in a body formed of a hydrogel matrix according to the present invention, the suspension of particulates is a suspension of two or more different types of particulates and the particulate substructure or structure of a layer is formed by said two or more different types of particulates, the two or more different types of particulates having identical, similar or preferably differing particulate distribution within said layer.

In a preferred embodiment of the process for the production of a three-dimensional particulate structure embedded in a body formed of a hydrogel matrix according to the present invention, the particulate substructure or structure of a layer is formed by subjecting the suspension of particulates in the layer of a hydrogel matrix precursor to a single vibration pulse. In general, in the context of the present invention, the duration of the pulse may be in the range of 5 to 60 seconds, more preferably of from 5 to 30 seconds. A suitable range of frequencies useful in the context of the present invention are frequencies of from about 10 Hz to 800 Hz.

In a preferred embodiment of the process for the production of a three-dimensional particulate structure embedded in a body formed of a hydrogel matrix according to the present invention, in at least one of the steps of forming a layer of a hydrogel matrix having a particulate substructure embedded therein, the concentration of particulates is increased or decreased with respect to any one of the previous steps

In a preferred embodiment of the process for the production of a three-dimensional particulate structure embedded in a body formed of a hydrogel matrix according to the present invention, in at least one of the steps of forming a layer of a hydrogel matrix having a particulate substructure embedded therein, the type of particulates is increased or decreased with respect to any one of the previous steps

In a preferred embodiment of the process for the production of a three-dimensional particulate structure embedded in a body formed of a hydrogel matrix according to the present invention, in at least one of the steps of forming a layer of a hydrogel matrix having a particulate substructure embedded therein, the type of cells is changed with respect to any one of the previous steps. For instance, if a skin implant is to be manufactured, it is possible to use cells in the lower layers of the skin implant which corresponds to the dermis and use keratinocytes and use cells in the lower layers of the skin implant which correspond to the epidermis and use fibroblasts. For instance, if an osteochondral implant is to be manufactured, it is possible to use cells in the lower layers of the osteochondral implant which corresponds to the bone region and use osteoblast and use cells in the upper layers of the osteochondral implant which correspond to the cartilage region and use chondrocytes.

In a preferred embodiment of the process for the production of a three-dimensional particulate structure embedded in a body formed of a hydrogel matrix according to the present invention, in at least one of the steps of forming a layer of a hydrogel matrix having a particulate substructure embedded therein, the type of hydrogel matrix is changed with respect to any one of the previous steps. For instance, if a skin implant is to be manufactured, it is possible to use a hydrogel matrix in the lower layers of the skin implant which corresponds to the dermis and use a hydrogel matrix comprising collagen and use a hydrogel matrix in the lower layers of the skin implant which corresponds to the epidermis and use a hydrogel matrix comprising collagen and keratin. For instance, if an osteochondral implant is to be manufactured, it is possible to use a hydrogel matrix in the lower layers of the osteochondral implant which corresponds to the bone region and use a hydrogel matrix composed by gelatine methacrylate, gelatine tyramine and use a hydrogel matrix in the upper layers of the osteochondral implant which corresponds to the cartilage region and use hyaluronan tyramine hydrogel.

The present invention allows to provide a quick and convenient way to obtain medical implants, as well as to obtain constructs that can be used for in vitro study of diseases and/or drug response, in particular such ones that have a relatively large volume.

### EXAMPLES

### Example 1

10 g of Type A gelatin, derived from porcine skin (Sigma-Aldrich) were dissolved in Dulbecco's phosphate buffered saline (DPBS) at 60° C. to make a 10 wt % uniform solution. To said solution 1.4 ml of methacrylic anhydride (MA) were added drop-wise under stirring conditions. The thus obtained mixture was allowed to react at 50° C. for 3 hours.

The resulting mixture was diluted 5-fold with additional warm DPBS and dialyzed against deionized water using a 12-14 kDa cutoff dialysis tube (VWR Scientific) for 6 days at 50° C. to remove unreacted methacrylic anhydride and additional by-products. After dialysis, the GelMA solution was filtered and frozen at −80° C. and subsequently lyophilized and stored at −20° C. until further use. The percent methacrylation of the gelatine was evaluated by NMR and found to be about 50%.

In order to obtain a suspension of cells and/or inorganic microparticles in GelMA solution, GelMA was dissolved in DMEM (or PBS) such as to yield a 10% w/v solution, to which 00.3% w/v of IRGACURE was added. Depending on the desired composition of the suspension, cells and/or inorganic microparticles were slowly added and gently mixed to form a suspension of cells and/or inorganic microparticles.

As an exemplary experiment, a three-layer construct was produced using three different layers containing different particulate patterns and/or particulates suspended therein were generated:

Layer 1 (FIG. 3 a, b)

By applying a vibrational motion having a frequency of 54 Hz and an amplitude of 4 V to 2 ml of GelMA/TCP microparticle suspension in a square petri dish (size: 30 mm×30 mm×5 mm) for approximately 10 to 15 seconds, a rounded checkerboard-like shape of TCP microparticles embedded in GelMA (FIG. 3 a, b) was obtained. The GelMA/TCP microparticle suspension was obtained by gently mixing 350 mg of TCP microparticles at 36° C. with 2 ml of GelMA. The layer was illuminated with a UV light source (5 mW/cm² for 40 s) in order achieve a partial crosslinking of about 80% of the GelMA.

Layer 2 (FIG. 3c)

By applying no vibrational motion to 2 ml of GelMA/iron oxide nanoparticles suspension in a square petri dish (size: 30 mm×30 mm×5 mm), a continuous and homogenous layer of iron oxide nanoparticles embedded in GelMA (FIG. 3 c) was obtained. The GelMA/iron oxide nanoparticles suspension was obtained by gently mixing 5 ml of iron oxide nanoparticles at 36° C. with 2 ml of GelMA. The layer was illuminated with a UV light source (5 mW/cm² for 40 s) in order achieve a partial crosslinking of about 80% of the GelMA.

Layer 3 (FIG. 3 f, h)

By applying a vibrational motion having a frequency of 77 Hz and an amplitude of 6 V to 2 ml of GelMA/TCP microparticle suspension in a circular petri dish ((diameter: 40 mm, thickness: 5 mm) for approximately 10 to 15 seconds, a concentric ring-like shape of TCP microparticles embedded in GelMA (FIG. 3 f, h) was obtained. The GelMA/TCP microparticle suspension was obtained by gently mixing 350 mg of TCP microparticles at 36° C. with 2 ml of GelMA. The layer was illuminated with a UV light source (5 mW/cm² for 40 s) in order achieve a partial crosslinking of about 80% of the GelMA.

After partially crosslinking each of the three layers, the layers were deposited on top of each other (bottom: Layer 1; Middle: Layer 2; Top: Layer 3) and exposed to further crosslinking radiation emanating from the UV light source illuminating the stack of layers with a UV light source (5 mW/cm² for 20 s) in order to fully crosslink the layers and bond them to each other. A schematic of the deposition is shown in FIG. 3.

### Example 2

TCP and resin in GelMA 5%

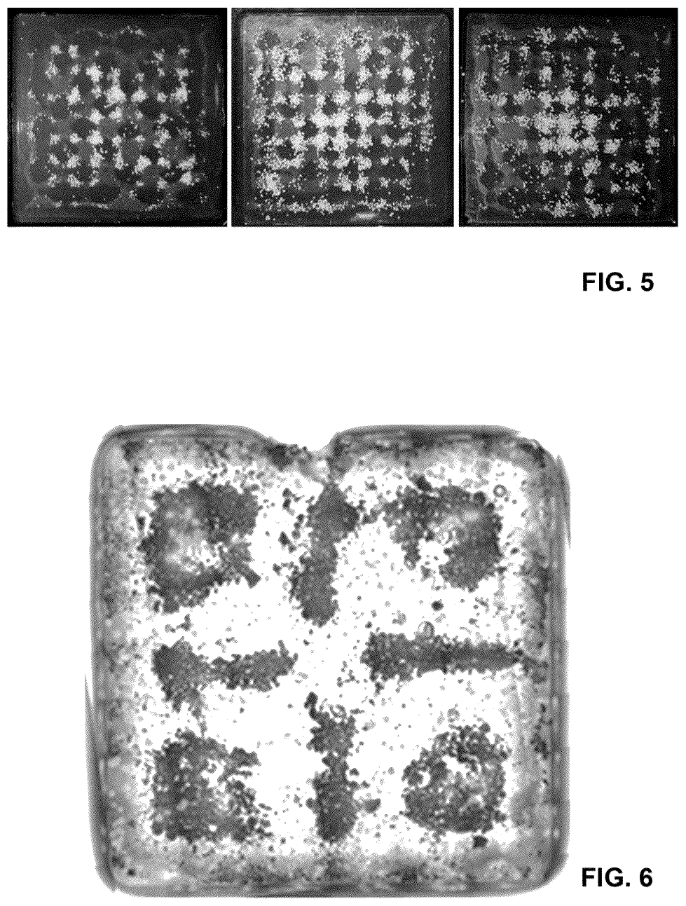

Two different types of particles were partitioned into different substructures. 20 mg of TCP particles having a diameter in the range of 32 to 75 μm and 20 mg of resin particles having a diameter in the range of 37 to 74 μm (Dowex 50W X8, Sigma-Aldrich) were suspended in 1 ml of GelMA 5% solution and loaded it into a square dish, and then exposed to a vibration of 60 Hz and allowed to solidify. The experiment was carried out in triplicate. FIG. 5 shows the resulting samples.

### Example 3

Two different types of particles were partitioned into different substructures. hMSC spheroids suspended in 2 ml of a fibrin gel were prepared and added to a square dish loaded with 70 mg of TCP particles having a diameter in the range of 250-500 μm. The spheroids and TCP particles were patterned together for about 10 to 15 s, and the fibrin gel was allowed to crosslink. The resulting body was cultured. The dual distribution of hMSC speroids and TPC particles can be seem in FIG. 6.

1. A process for the production of a three-dimensional particulate structure embedded in a body formed of a hydrogel matrix, comprising the steps of
a. forming a layer of a hydrogel matrix having a particulate substructure or structure embedded therein by
i. providing a suspension of particulates in a layer of a hydrogel matrix precursor in a container, said container having one or more inner surface portions vibrationally coupled to one or more vibration generators;
ii. subjecting the suspension of particulates in the layer of a hydrogel matrix precursor to a vibration emanating from one or more inner surface portions of the container vibrationally coupled to the vibration generator to cause standing acoustic waves in the hydrogel matrix precursor, thereby spatially partitioning the particulates within the layer of hydrogel precursor into a particulate substructure or structure, wherein the particulate substructure or structure is formed by subjecting the suspension of particulates in the layer of the hydrogel matrix precursor to one or more vibration pulse(s) at frequencies of from 10 Hz to 800 Hz;
iii. allowing the hydrogel precursor to solidify such as to form the layer of a hydrogel matrix having a particulate substructure or structure embedded therein,
further comprising the steps of:
b. forming a further layer of a hydrogel matrix having a particulate substructure embedded therein by carrying out step a. and depositing said further layer on top of the previously formed layer of a hydrogel matrix having a particulate substructure embedded therein, to form a three-dimensional particulate structure embedded in a body formed of a hydrogel matrix, and;
c. eventually repeating step b. at least one, two, three or more times such as to form a three-dimensional particulate structure embedded in a body formed of a hydrogel matrix, wherein in step a.iii, the hydrogel precursor is solidified by partially crosslinking the hydrogel precursor,
wherein a cross-linking agent is comprised in the hydrogel precursor layer as a mixture of the hydrogel precursor and a chemical crosslinking agent.

2. The process for the production of a three-dimensional particulate structure embedded in a body formed of a hydrogel matrix according to claim 1,
wherein in step a.iii, the hydrogel precursor is solidified by partially crosslinking the hydrogel precursor.

3. The process for the production of a three-dimensional particulate structure embedded in a body formed of a hydrogel matrix according to claim 1,
wherein in step a.iii), the hydrogel precursor is solidified by partially crosslinking the hydrogel precursor using a crosslinking agent.

4. The process for the production of a three-dimensional particulate structure embedded in a body formed of a hydrogel matrix according to claim 1,
wherein the hydrogel matrix comprises gelatine methacrylate, hyaluronic acid methacrylate, collagen, fibrin/thrombin, agarose, hyaluronan tyramine, gelatine tyramine, or alginate.

5. The process for the production of a three-dimensional particulate structure embedded in a body formed of a hydrogel matrix according to claim 1,
wherein the particulates are inorganic particulates, organic particulates, cells, aggregates of cells or cell spheroids.

6. The process for the production of a three-dimensional particulate structure embedded in a body formed of a hydrogel matrix according to claim 5,
wherein the inorganic particulates are capable of supporting bio-mineralization in an implant, and
wherein the organic particulates are capable of forming a scaffold in a medical implant.

7. The process for the production of a three-dimensional particulate structure embedded in a body formed of a hydrogel matrix according to claim 1,
wherein the particulate substructure or structure of a layer is formed such as to have a differing particulate distribution from, a particulate substructure or structure of another layer.

8. The process for the production of a three-dimensional particulate structure embedded in a body formed of a hydrogel matrix according to claim 1,
wherein the suspension of particulates is a suspension of two or more different types of particulates and the particulate substructure or structure of a layer is formed by said two or more different types of particulates, the two or more different types of particulates having differing particulate distributions within said layer.

9. The process for the production of a three-dimensional particulate structure embedded in a body formed of a hydrogel matrix according to claim 1,
wherein the particulate substructure or structure of a layer is formed by subjecting the suspension of particulates in the layer of a hydrogel matrix precursor to a one vibration pulse.

10. The process for the production of a three-dimensional particulate structure embedded in a body formed of a hydrogel matrix according to claim 1,
wherein the standing acoustic waves produced in the hydrogel matrix precursor to spatially partition the particulates within the layer of hydrogel precursor into a particulate substructure are modified in between steps of forming layers of a hydrogel matrix having a particulate substructure embedded therein.

11. The process for the production of a three-dimensional particulate structure embedded in a body formed of a hydrogel matrix according to claim 1,
wherein within at least one of the steps of forming a layer of a hydrogel matrix having a particulate substructure embedded therein, the concentration of particulates is increased or decreased in between steps of forming layers of a hydrogel matrix having a particulate substructure embedded therein.

12. The process for the production of a three-dimensional particulate structure embedded in a body formed of a hydrogel matrix according to claim 1,
wherein within at least one of the steps of forming a layer of a hydrogel matrix having a particulate substructure embedded therein, the type or concentration of particulates is modified in between steps of forming layers of a hydrogel matrix having a particulate substructure embedded therein.

13. The process for the production of a three-dimensional particulate structure embedded in a body formed of a hydrogel matrix according to claim 1,
wherein within at least one of the steps of forming a layer of a hydrogel matrix having a particulate substructure embedded therein, the type of hydrogel matrix is modified in between steps of forming layers of a hydrogel matrix having a particulate substructure embedded therein.

14. The process for the production of a three-dimensional particulate structure embedded in a body formed of a hydrogel matrix according to claim 1, wherein the process further includes a step of:
b. crosslinking the deposited layers of hydrogel matrix having a particulate substructure embedded therein such as to form a three-dimensional particulate structure embedded in a body formed of a hydrogel matrix.

15. A three-dimensional particulate structure embedded in a body formed of a hydrogel matrix wherein said structure is obtained by a process according to claim 1.

16. The process for the production of a three-dimensional particulate structure embedded in a body formed of a hydrogel matrix according to claim 3, wherein in step a.iii), the hydrogel precursor is solidified by partially crosslinking the hydrogel precursor using a crosslinking agent capable of being activated by a physical stimulus or a chemical stimulus.

17. The process for the production of a three-dimensional particulate structure embedded in a body formed of a hydrogel matrix according to claim 3,
wherein in step a.iii), the hydrogel precursor is solidified by partially crosslinking the hydrogel precursor using radiation or temperature.

18. The process for the production of a three-dimensional particulate structure embedded in a body formed of a hydrogel matrix according to claim 3,
wherein in step a.iii), the hydrogel precursor is solidified by partially crosslinking the hydrogel precursor using enzymes, pH change or ion concentration change.

19. The process for the production of a three-dimensional particulate structure embedded in a body formed of a hydrogel matrix according to claim 7,
wherein the particulate substructure or structure of a layer is formed such as to have an identical or similar particulate distribution as a particulate substructure or structure of another layer.

20. The process for the production of a three-dimensional particulate structure embedded in a body formed of a hydrogel matrix according to claim 8,
wherein the suspension of particulates is a suspension of two or more different types of particulates and the particulate substructure or structure of a layer is formed by said two or more different types of particulates, the two or more different types of particulates having identical or similar or particulate distributions within said layer.

21. The process for the production of a three-dimensional particulate structure embedded in a body formed of a hydrogel matrix according to claim 5,
wherein the particulates are cells, and the cells are osteoblasts, fibroblasts, keratinocytes, human mesenchymal stem cells (hMSCs), chondrocytes, human umbilical vein endothelial cells (hUVECs), or aggregates of said cells or cell spheroids of said cells.

22. The process for the production of a three-dimensional particulate structure embedded in a body formed of a hydrogel matrix according to claim 6,
wherein the inorganic particulates are hydroxyapatite or calcium phosphate particulates and the organic particulates are polylactic acid or polyhydroxybutyric acid.
